(19) United States
(12) Patent Application Publication
(10) Pub. No.: US 2002/0160408 A1
(43) Pub. Date: Oct. 31, 2002

(54) 2ER membrane and lumenal protein interaction yeast two-hybrid assay

(75) Inventors: Marc F. Pelletier, Branford, CT (US); Gregor Jansen, Montreal (CA); John J.M. Bergeron, Pointe-Claire (CA); David Y. Thomas, Montreal-West (CA)

Correspondence Address: OGILVY RENAULT, 1981 MCGILL COLLEGE AVENUE, SUITE 1600, MONTREAL, QC, H3A2Y3, CA

(73) Assignee: NATIONAL RESEARCH COUNCIL OF CANADA, Ottawa, ON (CA)

(21) Appl. No.: 10/118,937

(22) Filed: Apr. 10, 2002

Related U.S. Application Data
(60) Provisional application No. 60/282,854, filed on Apr. 11, 2001.

(30) Foreign Application Priority Data
Oct. 9, 2001 (CA) 2,358,758

(51) Int. Cl. C12Q 1/68; C12N 1/18; C12N 15/74

(52) U.S. Cl. 435/6; 435/254.2; 435/483

(57) ABSTRACT

The present invention relates to a new yeast two-hybrid system for the detection of interactions between membrane proteins and lumenal proteins of the endoplasmic reticulum. The yeast two-hybrid system of the present invention uses the IRE1 gene of yeast which is a key element in the endoplasmic reticulum unfolded protein response to signal the interaction between proteins within the endoplasmic reticulum. The IRE1 gene codes for a type 1 membrane protein Ire1p, that has a kinase in the cytosolic domain and it signals the presence of unfolded proteins in the ER by oligomerization and transphosphorylation, this in turn activates a RNaseL that has as its unique (so far) substrate the HAC1 messenger RNA, that codes for the transcription factor that binds to the unfolded protein response element and upregulates transcription of ER protein required for folding proteins within the ER. The yeast two-hybrid system of the present invention uses in-reading frame fusions of ER proteins to the N-terminal “protein sensing domain” of Ire1p to detect their interaction using Ire1p dimerization and the unfolded protein response system as a read out. This readout is made simpler by the use of reporter gene systems making the system suitable for mass screening of ER protein interactions.

### BACKGROUND OF THE INVENTION

1. Field of the Invention

The invention relates to the detection of interactions between proteins in the membrane and/or lumen of the endoplasmic reticulum (ER) of eukaryotic cells using a yeast-based two-hybrid system.

2. Description of Prior Art

Yeast two hybrid systems derive from the two hybrid protein interaction assay developed by Fields and Song (Nature 340, 245-246 (1989)) and subsequent variants and improvements. Using these systems a plethora of intracellular protein-protein interactions have been found and characterized. The elegant simplicity of this genetically based-system supplemented biochemical approaches to studying protein-protein interactions. It is the technology that permits the rapid identification of binding partners, the definition of residues critical to the interaction, and more recently the construction of large scale protein interaction networks. These are however, composed of soluble cytosolic and nuclear proteins with some interactions with the cytosolic domains of membrane proteins. Regardless of the inherent constraints of the classical yeast two-hybrid system, it has been selected for the formidable endeavor of comprehensively mapping the protein-protein interactions within the nematode Caenorhabditis elegans (Walhout, A. J., Boulton, S. J. & Vidal, M., Yeast 17, 88-94 (2000)) and yeast Saccharomyces cerevisiae (Uetz, P. et al., Nature 403, 623-627 (2000); Schwikowski, B., Uetz, P. & Fields, S. Nature Biotech. 18, 1257-1261 (2000)) for which the complete genomes are now available (Goffeau A et al., Science 274, 543-547 (1996); Science 282, 2012-2018 (1998)). There are some two-hybrid systems that report some interactions with membrane proteins. These include the split-ubiquitin system (Stagljar, I. et al, Proc. Natl. Acad. Sci. USA 95, 5187-5192 (1998); Johnsson, N. & Varshavsky, A., EMBO J. 13, 2686-2698 (1994)), the SOS and Ras recruitment systems (SRS and RRS) (Aronheim, A. et al., Mol. Cell Biol, 17, 3094-3102 (1997); Broder, Y. C., Katz, S. & Aronheim, A, Curr. Bio. 8, 1121-1124 (1998)), G-protein fusions (Ehrhard KN et al., Nature Biotech. 18, (2000)), and the oligomerization-assisted enzymatic complementation systems which result in the reassembly of murine dihydrofolate reductase (mDHFR) (Pelletier, J. N. et al., Nature Biotech. 17, 683-690 (1999)) or the β-galactosidase (Rossi, F. M., Blakely, B. T. & Blau, H. M., Trends Cell Biol 10, 119-122 (2000)) from E. coli. These techniques have broadened the spectrum of proteins that can be analyzed, to include systems to study transcriptional activators that tend to self-activate the Gal4p based system (Aronheim, A. et al., Mol. Cell Biol, 17, 3094-3102 (1997)), integral membrane and membrane-associated proteins which are topologically restricted from the nucleus (Stagljar, I. et al, Proc. Natl. Acad. Sci. USA 95, 5187-5192 (1998); Broder, Y. C., Katz, S. & Aronheim, A., Curr. Bio. 8, 1121-1124 (1998);Ehrhard K N et al., Nature Biotech. 18, (2000)), and also permitted the study of protein-protein interactions directly in mammalian cells.

It would be highly desirable to be provided with a system that could report reliably and with high throughput the interaction of membrane proteins and specifically membrane and lumenal proteins of the endoplasmic reticulum.

### SUMMARY OF THE INVENTION

One aim of the present invention is to provide a new system for the detection of specific protein-protein interactions in the endoplasmic reticulum that uses the unique properties of a type 1 membrane protein encoded by the yeast gene IRE1. This protein termed Ire1p has an N-terminal signal sequence, an “unfolded protein sensing domain” in the lumen of the endoplasmic reticulum, a transmembrane domain, a cytoplasmic serine/threonine kinase domain, and a C-terminal RNaseL domain. Fusions of proteins that interact in the endoplasmic reticulum behind the signal sequence and in front of the transmembrane domain make possible the detection of their interactions.

In accordance with the present invention there is provided a yeast two-hybrid assay for detection of interactions between at least two endoplasmic reticulum membrane and/or lumenal proteins of interest capable of or suspected of interacting, which comprises:

recombinational cloning of necessary DNA elements in a specially constructed plasmid in a reporter yeast strain wherein said plasmid is capable of expressing fusion proteins each comprising a fusion of a protein of interest and transmembrane domain and C-terminal kinase and RnaseL domains of yeast Ire1p kinase (unfolded protein response signaling kinase), wherein said reporter yeast strain having a reporter gene integrated and a Δire1 genotype, wherein said reporter gene is controlled by an unfolded protein response (UPR) element, and wherein said reporter yeast strain non-recombined is incapable of an unfolded protein response (UPR); and

monitoring expression of said reporter gene by activation of an unfolded protein response (UPR), wherein said expression is indicative of protein interaction.

The reporter gene may be LacZ gene from E. coli.

The reporter gene may be under the control of a promoter having an unfolded protein response element upstream, and wherein said reporter is selected from the group consisting of HIS3, URA3, another yeast gene having a visible growth phenotype and a coloured substrate indicating transcription and translation of the reporter gene in response to oligomerization of Ire1p.

The promoter may be a chimeric promoter comprising a minimal yeast unfolded protein response element (UPRE) and a truncated CYC1 promoter.

The protein interactions may be occurring within an intracellular organelle, such as the endoplasmic reticulum (ER) or its lumen.

The protein may be a membrane protein or a soluble protein.

The protein may also be a glycoprotein.

The yeast may be Saccharomyces cerevisiae.

In accordance with the present invention there is provided a method for mapping protein interactions, which comprises using the yeast two-hybrid assay of the present invention.

The regions of protein-protein interaction of calnexin and calreticulin with Erp57 may be mapped.

In accordance with the present invention there is provided a high throughput assay for detection of protein interaction networks, which comprises the assay of the present invention combined with high throughput technology.

In accordance with the present invention there is provided a method for the analysis of protein interaction networks of the ER may be effected using the high throughput assay of the present invention.

### BRIEF DESCRIPTION OF THE DRAWINGS

FIG. 1 illustrates a model for a yeast-ER two-hybrid system based on the UPR sensor, Ire1p. Chimeric fusions of proteins (for example X and Y) with the transmembrane kinase/endoribonuclease Ire1p that lead to oligomerization, consequently induce transphosphorylation of the Ire1p kinase domain, nuclear targeting of the Ire1p endoribonuclease domain, that then processes the mRNA of the UPR transcriptional activator, Hac1p. With the intron removed, Hac1p is expressed as the active form (Hac1p₁) which binds to the minimal yeast unfolded protein response (UPR) element upstream of the bacterial LacZ gene.

FIGS. 2A-C illustrate interactions between calnexin and calreticulin, with ERp57, using the Ire1p based two-hybrid system, and functional complementation of the lumenal domain of Ire1p, by chimeric fusions with Ire1p that leads to dimerization of the fused proteins. (A) Schematic diagram of the constructs used to test the yeast-ER two-hybrid system are shown. The parental vectors pLJ89 (LEU2) and pLJ96 (HIS3) encode the Ire1p signal sequence (SS), a 23 aa linker (L), in frame with the Ire1p transmembrane (TM) and kinase/endoribonuclease domains, under the control the IRE1 promoter. Clone accession numbers are indicated in parentheses. (B) β-galactosidase (LacZ) filter assay was performed on S. cerevisiae diploid strains expressing Ire1p fusion proteins. Yeast cell patches prior to transfer are shown on right. Crosses were made with strains carrying pLJ89 and pLJ96 as negative controls, while the extracellular domain of the murine erythropoietin receptor (EPOr) served as a positive control. (C) Quantitative permeable cell/β-galactosidase assays were performed on yeast cells in the absence or presence of 5 μg/ml of tunicamycin for 1 h.β-galactosidase activity is reported in the absence of tunicamycin (lightly shaded bars) and for treated cells (dark bars). Plasmid combinations are indicated, and the parental W303a strain (with the integrated reporter) was used as a positive control (W+).β-galactosidase units are defined as (A₄₂₀×1000)/(A₆₀₀ of cells×culture vol.(ml)×reaction time (min)).

FIGS. 3A-B illustrate the loop domains of calnexin and calreticulin are sufficient, and the B thioredoxin domain of ERp57 is required for the dimerization of both calnexin and calreticulin, with ERp57. (A) Schematic diagram of the constructs used to map the protein-protein interaction domains of calnexin (CNX), calreticulin (CRT), and ERp57. The loop domains of CNX and CRT, with their corresponding type 1 and type 2 repeat configuration, and the thioredoxin domains of ERp57 (A,B,B′,A′) are shown. (B) β-galactosidase filters assay on strains expressing the mapping fusion proteins described in A. pLJ89/pLJ96 and EPOr were used as negative and positive controls, respectively.

FIGS. 4A-B illustrate the loop domain of calnexin interacts directly with ERp57 in vitro. (A) GST fusions consisting of the full length lumenal domain of CNX (CNX_{K46-M417}, lane 1), the loop domain (or P-region, CNX_{M267-L412}, lane 2), and GST alone (lane 3) were purified and are shown on a 10% SDS-PAGE. (B) The purified fusion proteins were loaded onto columns with Glutathione Sepharose 4B, followed by purified ERp57 (Zapun, A. et al., J. Biol. Chem. 273, 6009-6012 (1998)). The columns were washed, the proteins were then eluted with reduced glutathione, and a Western blots performed on the eluant with anti-ERp57 antiserum.

### DETAILED DESCRIPTION OF THE INVENTION

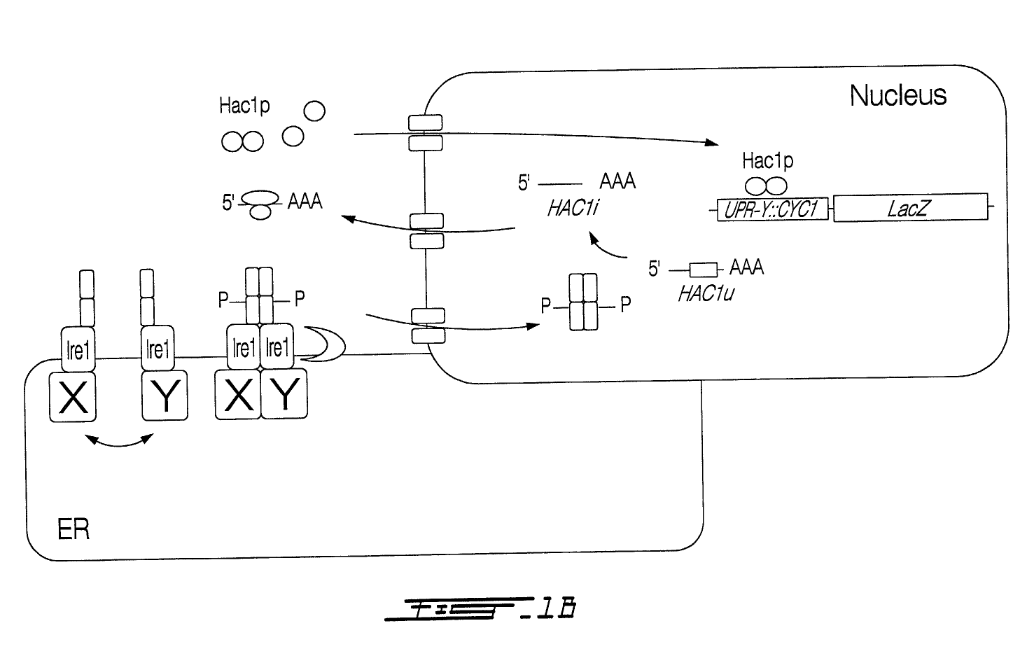

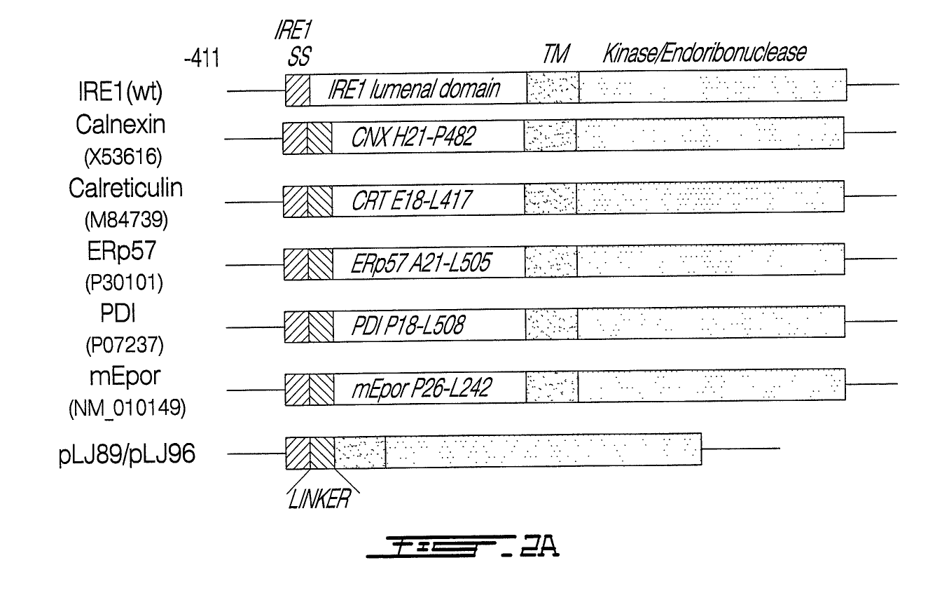

In accordance with the present invention, there is provided a yeast two-hybrid system based on the use of the unfolded protein response (UPR) in Saccharomyces cerevisiae, to signal dimerization of chimeric fusion proteins of (FIG. 1B). Fusions were made with Ire1p, an ER transmembrane kinase/endoribonuclease that normally oligomerizes upon the detection of accumulated misfolded proteins within the ER20. Upon oligomerization, the kinase domains auto-transphosphorylate, a prerequisite for the activation of the endoribonuclease domain (Shamu, C. E. & Walter, P., EMBO J. 15, 3028-3039 (1996)). The endoribonuclease domain is then targeted to the nucleus. While it has been shown for the mammalian homologues of IRE1p, termed Ire1α and Ire1β (Wang, X. Z. et al., EMBO J. 17, 5708-5717 (1998); Tirasophon, W., Welihinda, A. A. & Kaufman, R. J., Genes Dev. 12, 1812-1824 (1998)), that the endoribonuclease domain undergoes cleavage in a presenilin (PS1) dependent manner prior to nuclear targeting (Niwa, M. et al., Cell 99, 691-702 (1999)), it is unclear whether such a process, including the nuclear targeting of the endoribonuclease domain actually occurs in yeast. Nevertheless, the endoribonuclease domain of Ire1p then processes HAC1 mRNA24. Hac1p, a bZIP transcriptional activator that binds the UPR element (UPRE), regulates the expression of many molecular chaperones, and folding enzymes of the ER, as well as many components of the ER associated degradation pathway (ERAD) (Travers, K. J. et al., Cell 101, 249-258 (2000)). The intron within HAC1 mRNA has been shown to attenuate the translation of this mRNA (Chapman, R. E. & Walter, P., Curr. Biol. 7, 850-859 (1997)). We integrated a cassette with the LacZ gene from E. coli, under the control of a chimeric promoter with the minimal yeast UPR element fused to a truncated CYC1 promoter as a reporter, into Saccharomyces cerevisiae (W303a). This strain was then crossed with BY4742 (Δire1), and haploids were isolated that carried both the integrated reporter with the ΔIRE1 genotype.

We have used some elements of the UPR to construct the system of the present invention that reports protein interactions in the ER. To use this system, we first integrated a cassette with the LacZ gene from E. coli under the control of a chimeric promoter into Saccharomyces cerevisiae (W303a). This promoter consists of a minimal yeast UPRE fused to a truncated CYC1 promoter. The reporter strain was then created by crossing this strain with BY4742 (Δire1), and haploids were isolated that carried both the integrated reporter with the Δire1 genotype. This strain cannot respond with a UPR to agents that induce this response in the wild-type.

Th ER protein two-hybrid system of the present invention provides a tool for mapping protein interactions for both membrane (as is the case for CNX) and soluble ER proteins within the ER, and potentially in other organelles. There is some evidence that the S. cerevisiae CNX (Cne1p) and UGGT (Kre5p) homologues have different functions from their mammalian homologues, which may explain why the heterologously expressed mammalian ER proteins do not appear to be interfering with their yeast counterparts³¹. However, the yeast ER does provide the redox potential, calcium and ionic concentrations, and some of the enzymes and chaperones that are found in mammalian cells enabling the specific interactions to be detected.

The ER protein two-hybrid system is, in conjunction with our recombinational cloning approach, simple and amenable to high throughput technology, and hence could be used for the comprehensive analysis of the protein interaction networks of the ER.

### Experimental Protocol

Manipulations, Yeast Strains, and Plasmids

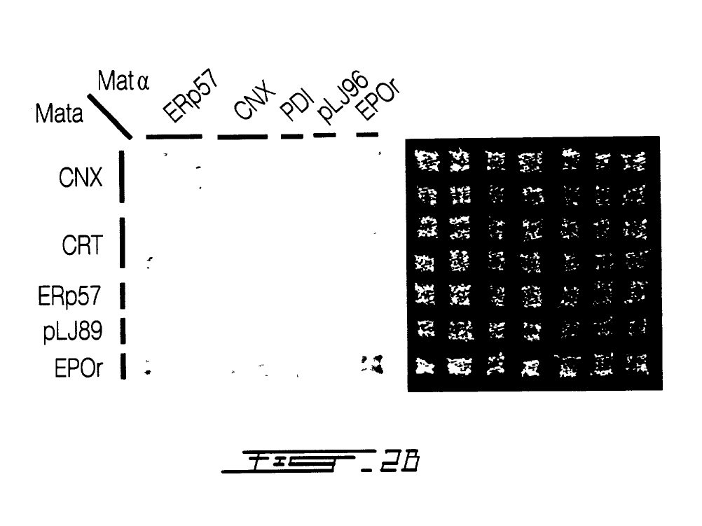

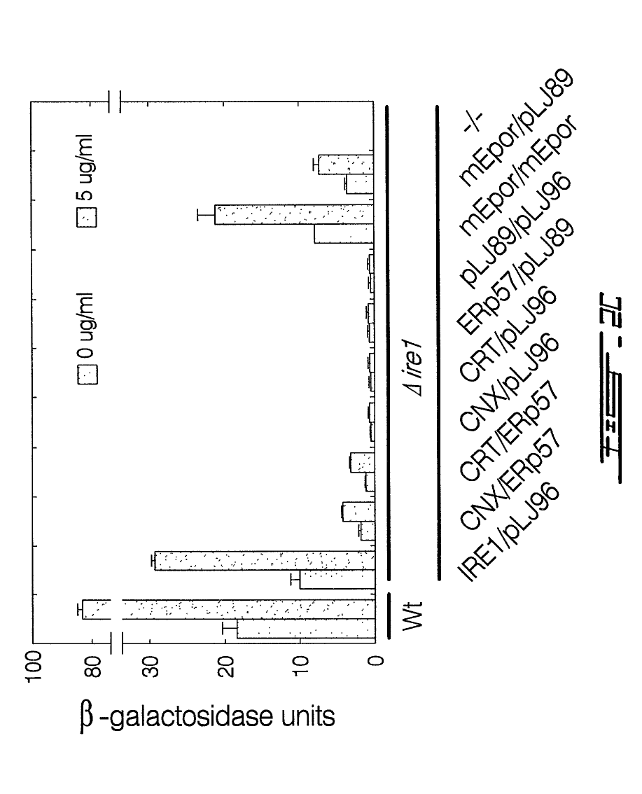

Standard protocols were used for yeast growth and transformations (Guthrie, C. & Fink, G. R. Guide to yeast genetics and molecular biology. In Academic Press. San Diego (1991)). The reporter strain was constructed by integrating UPR-Y::CYC1::LacZ, derived from pLG-Δ178 (Mori, K. et al., EMBO J. 11, 2583-2593 (1992)) kindly provided by Claude A. Jakob (Zurich, Switzerland) into the ura3-52 locus of W303a (MATa; ura3-52; trp1; leu2; his3; ade2; can1-100). This strain was then crossed to BY4742 Matα ΔIRE1::KanMX (ATCC #4011907). The diploids were sporulated, and haploids of both mating types were isolated (yLJ29 MATa; trp1; leu2; his3; ΔIRE1::KanMX; ura3-52::UPR-Y::LacZ-URA3, and yLJ31 MATα ura3; trp1; leu2; his3; ade2; ΔIRE1::KanMX; UPRY::CYC1::LacZ). Unless otherwise mentioned, all plasmids were made by recombinational cloning directly in yeast as described elswhere (Uetz, P. et al., Nature 403, 623-627 (2000)). The parent plasmid pLJ89 was made in three steps: (1) a 2 μ yeast plasmid with a LEU2 marker (pGreg505) was linearized and recombined with a 477 bp PCR product (amplified using Expand High Fidelity™ system (Roche, Laval, Canada)) that spanned from −411 to +66 bp coding region of IRE1 (i.e. IRE1 promoter and ER signal sequence). (2) A 66 bp linker that has both Notl and Safl sites was added 3′ to the sequence encoding the ER signal sequence, without disrupting the reading frame. (3) The region encoding the transmembrane domain, kinase, and endoribonuclease was amplified and was similarly introduced (FIG. 2A). To produce pLJ96, the IRE1 cassette was removed from pLJ89 by Pmel and Xbal, (New England Biolabs, Mississauga, Canada) and subcloned into pGreg503, that contains a HIS3 marker. All constructs used for testing protein interactions were derived from pLJ89 and pLJ96. They were linearized with Safl, the genes of interest amplified by high fidelity PCR with, then recombined into pLJ89 or pJ96, in the strains yLJ29 or yLJ31.

β-Galactosidase Assays

Filter assays were performed on strains that were grown for two days, mated on YPD for 12 hours, and diploids isolated and grown on synthetic dropout medium (-Ura -Leu -His) for 24 to 48 hours. The cell patches were transferred to nitrocellulose membrane, dipped in liquid N₂ for 5 seconds, and then placed on Whatman filter paper soaked in Z buffer (60 mM Na2Hpo4·7H2O, 40 mM NaH2PO4.H2O, 10 mM KCI, 1 mM MgSO4·7H2O, 50 mM β-mercaptoethanol, pH7.0) with 4 mg/ml X-Gal. The filters were then incubated for 30-60 minutes at 30° C. Quantitative β-galactosidase assays were performed as described elsewhere (Nantel, A., Mohammad-Ali, K., Sherk, J., Posner, B. I. & Thomas, D. Y., J. Biol. Chem. 273, 10475-10484 (1998)) and repeated three times, each with n=3.

Production of GST Fusion Proteins and GST-pull Down Assay

Plasmids encoding GST-CNX_{K46-M417} and GST- CNX_{M267-L212} were a kind gift from Robert Larocque (Montreal, Canada). Expression and purification of the GST fusions were performed as previously described (Zheng, C. F. & Guan, K. L., J. Biol. Chem. 268, 23933-23939 (1993)). Approximately 2 μg of GST-CNX_{K46-M417}, GST- CNX_{M267,L212}, or GST were loaded onto a column with 200 μl of Glutathione Sepharose 4B (Amersham Pharmacia Biotech, Piscataway, N.J.) pre-equilibrated with TBS 1% Triton X-100 pH 7.0. The resin was washed with 5 ml of the same buffer, and 5 μg of ERp57 was loaded onto the resin. The column was washed again with 10 ml of TBS, and then the proteins were eluted in 100 μl of 10 mM reduced glutathione. Western analysis was performed as described elswhere (Pelletier, M. F. et al., Glycobiology 10, 815-827 (2000)), with rabbit polyclonal anti-ERp57 antiserum.

The present invention will be more readily understood by referring to the following example which is given to illustrate the invention rather than to limit its scope.

### EXAMPLE I

The parental plasmids, pLJ89 and pLJ96 (FIG. 2A), with LEU2 and HIS3 markers respectively, encode Ire1p with the lumenal domain deleted to the transmembrane domain (TM). The Ire1p signal sequence is intact and followed by a 23 amino acid linker to ensure that the signal peptidase does not cleave the nascent protein subcloned in place of the Ire1p ER lumenal domain. The genes of interest were amplified by high fidelity PCR, and subcloned directly into yeast by recombinational cloning between the encoding regions of the linker and the TM domains. To test the approach, MATa strains expressing the first set of fusions with the lumenal domain of calnexin, calreticulin, ERp57, the parental vector pLJ89 (negative control), and the extracellular domain of the murine erythropoietin receptor (EPOr, positive control), respectively, were streaked and then mated to MAT strains expressing the second set, thus ERp57, the lumenal domain of calnexin, the ER protein disulphide isomerase (PDI), the parental vector pLJ96 (negative control), and again the extracellular domain the EPOr (positive control), respectively (FIG. 2B). Diploid strains expressing both sets of fusion proteins were isolated, transfered to nitrocellulose, and tested for β-galactosidase activity. Using a matrix appoach, it was shown as previously by functional assay and crosslinking (Oliver, J. D. et al., Mol Biol. Cell. 10, 2573-2582 (1999)), that calnexin (lumenal domain) and calreticulin interact specifically with ERp57 in vivo, and not with PDI, its sequence related homologue. Also, when the alternative combination was used to subclone calnexin and ERp57 into the parental vectors pLJ89 and pLJ96, the interaction was still observed. Importantly, calnexin did not interact with calreticulin, nor did ERp57 interact with PDI, nor was dimerization observed for these fusion proteins. We used the extracellular domain of the murine erythropoietin receptor as a positive control, as it is known to normally dimerize. Ligand binding to the EPOr does not affect its dimerization, but causes a change in orientation of the cytosolic JAK-2 kinase domains, which in turn are activated through trans-phosporylation (Livnah, O. et al, Nat. Struct. Biol. 5, 993-1004 (1998); Remy, I., Wilson, I. A. & Michnick, S. W., Science 283, 990-993 (1999); Wilson, I. A. & Jolliffe, L. K., Curr. Opin. Struct. Biol. 9, 696-704 (1999)). Our results confirm this.

A ligand-independent activation model for Ire1p was recently proposed by Liu et al. (Liu, C. Y., Schroder, M. & Kaufman, R. J., J. Biol. Chem. 275, 24881-24885 (2000)) They found that a leucine zipper dimerization motif could replace the function of the lumenal domain of Ire1p, in sensing an accumulation of unfolded proteins within the ER. We tested whether this was also true for fusions that lead to oligo- or dimerization of Ire1p chimeras. This would provide supportive evidence that the fusion proteins are indeed in the proper topological orientation and localization. Quantitative β-galactosidase assays were performed on diploid cells from the previous experiment. They were grown to mid-log phase and treated for one hour with 5 μg/ml of tunicamycin, which inhibits N-linked glycosylation inducing the unfolded protein response. In both cases, either dimerization, the UPR was activated, consequently leading to an increase in expression of our reporter, when compared to the control strains. This supports the findings of Liu et al. (Liu, C. Y., Schroder, M. & Kaufman, R. J., J. Biol. Chem. 275, 24881-24885 (2000)) that ligand dependent dimerization of Ire1p activates UPR. It also suggests that (1) chimeric fusions that lead to both homo- and heterodimerization will functionally complement Ire1p, and (2) our chimeric fusions are localized to the endoplasmic reticulum. Interestingly, a dose dependent response was seen for the diploid strains expressing the murine EPOr, with a response approaching that of the strain expressing Ire1p (wild-type). We should note that the tunicamycin induced UPR in the strain with the plasmid born Ire1p was however, weaker than in the parental W303a strain. It is possible that 411 bp 5′ of the ATG codon of IRE1 is insufficient as a promoter to express a full complement of IRE1, or alternatively, the genotype of the ire1 reporter strains leads to a slightly weaker UPR than for the W303a parental strain. To confirm that these parental vectors target the fusion proteins to the endoplasmic reticulum, we subcloned GFP as the lumenal domain, under the control of a GAL1 promoter. Even under the conditions of high levels of expression, the fusion protein displayed a perinuclear localization typical of ER membrane proteins in S. cerevisiae.

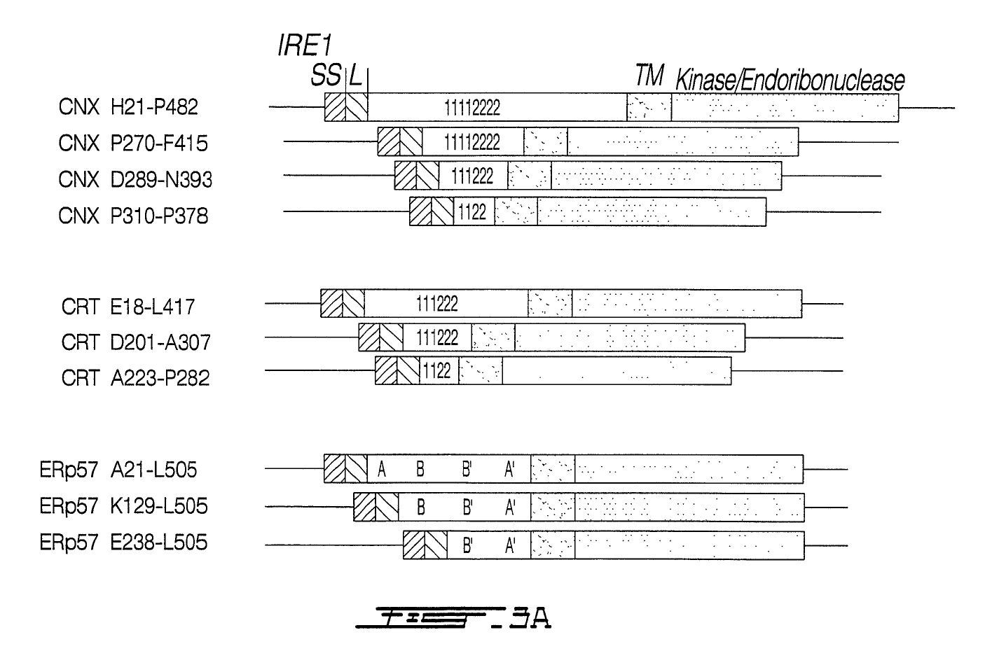

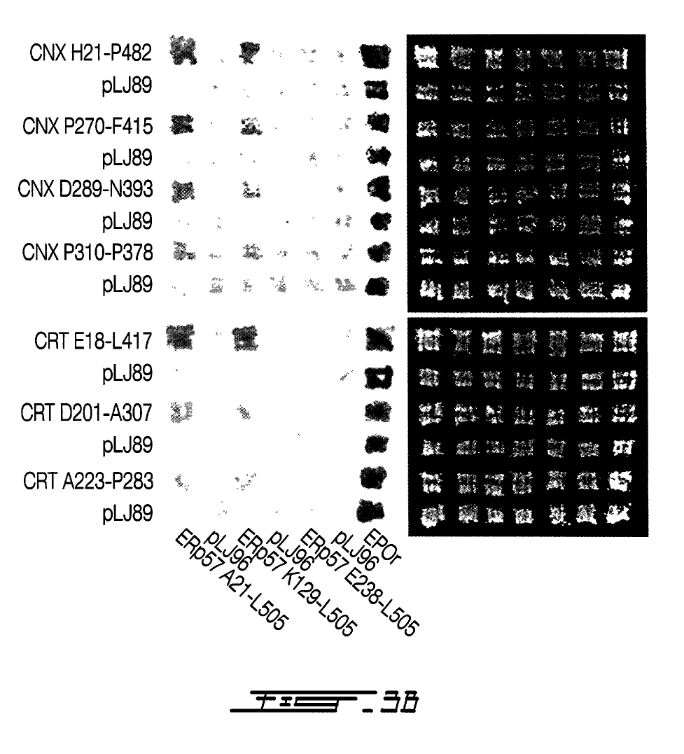

Next, we set out to map the protein-protein interactions between calnexin and calreticulin, with ERp57. The structure of calnexin is known at a 2.9 Å resolution (Schrag, J. D et al., MOL. CELL 8: 633-644 (2001)), and is characterized by two main structural components: a globular lectin domain and a long “arm” loop domain. This loop, also known as the P-domain for its proline-rich primary sequence (Wada, I. et al., J. Biol. Chem. 266, 19599-19610 (1991); Baksh, S. & Michalak, M., J. Biol. Chem. 266, 21458-21465 (1991)) branches out of the lectin domain at residue P270, with four copies of a repeat motif (type 1), and then returns intertwining back to the lectin domain at residue F₄₁₅, with four copies of another repeat motif (type 2) in a “11112222” configuration, forming a hook-like arm. The topology of the calnexin loop domain is in agreement with that of calreticulin, proposed by Ellgaard et al. (Ellgaard, L. et al., FEBS Lett. 488, 69-73 (2001)) The calreticulin loop is however shorter than that of calnexin: it is missing the first type 1 repeat, as well as the last type 2 repeat, and thus resembles a shortened calnexin loop with a “111222” configuration. When tested in our two-hybrid system, the calnexin lectin domain failed to mediate a specific interaction with ERp57, while its loop domain (P₂₇₀-F₄₁₅) did confer this specificity. We therefore designed mapping constructs for the loop domains of calnexin and calreticulin (FIG. 3A). To summarize, in addition to the full lumenal domains of calnexin and calreticulin, we made three additional calnexin, and two additional calreticulin mapping constructs. We subcloned (1) the region encoding the tip of the calnexin loop, which contains two of each repeat motif (repeats 1122, P₃₁₀-P₃₇₈), (2) the longer loop with three of each repeat (111222, D₂₈₉-N₃₉₃), (3) and one which has four of each and forms the entire loop domain (11112222, P₂₇₀-F₄₁₅) (FIG. 3A). Similarly for calreticulin, we subcloned the tip region (1122, A₂₂₃-P₂₈₃), and the full calreticulin loop (111222, D₂₀₁-A₃₀₇) (FIG. 3A).

The mapping constructs for ERp57 were based on the proposed domain structure of its sequence-related homologue PDI36. Both PDI and ERp57 contain four thioredoxin domains in tandem (A—B—B′—A′). The A and A′ share high similarity to thioredoxin, and each contain a copy of the active site consensus sequence —C—G—H—C—, with the N-terminal cysteine being reactive, while the B and B′ domains, which are less conserved, have lost this active site consensus sequence36. Again, in addition to the full-length ERp57 fusion construct, we made two deletion constructs with (1) the first thioredoxin domain removed (B—B′—A′, K₁₂₉-L₅₀₅), and the second, with the first two removed (B′—A′, E₂₃₈-L₅₀₅) (FIG. 3A). To test the protein-protein interactions, the calnexin and calreticulin constructs were transformed into the MATa reporter strain, while the ERp57 constructs, into the Matα reporter strain. The strains were streaked and crossed in the form of a matrix, and the interactions were verified by β-galactosidase filter assay (FIG. 3B). The parental plasmids pLJ89 and pLJ96, and the extracellular domain of EPOr were used as negative and positive controls, respectively. This experiment showed that two sets of repeat motifs, hence the tip of loop domain (with a “1122” repeat configuration) is sufficient to mediate the interaction of both calnexin and calreticulin, with ERp57. Moreover, the second thioredoxin domain of ERp57 (B) is required to interact with both lectin-like chaperones. This further confirms the specificity of our system, and demonstrates the sensitivity by which small domains (60 aa in the case of calreticulin) can be used for testing proteinprotein interactions. It also suggests a role for the loop domains of calnexin and calreticulin, as well as the non-catalytic B domain of ERp57 in mediating oligomerization.

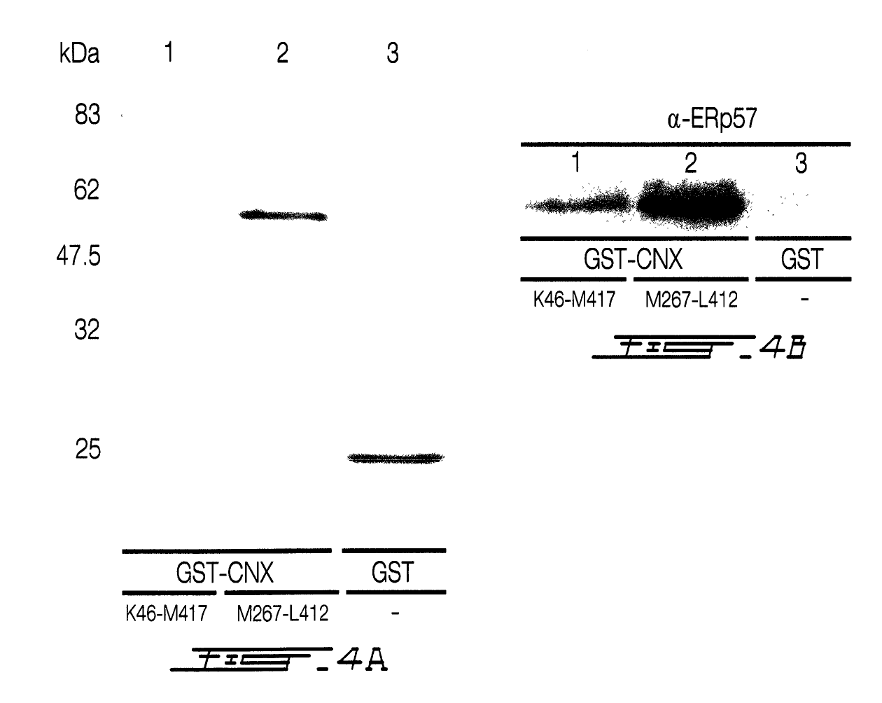

To confirm that these two-hybrid results can also be seen as a physical interaction, we used a GST fusion of the full lumenal domain of CNX (GST-CNX_{K46-M417}), and the loop domain (GST-CNX_{M267-L412}), using GST as a control, and tested for ERp57 binding (FIG. 4A). The results show that ERp57 binds specifically to both GST-CNX fusions (FIG. 4B). Thus the functional (3) and crosslinking (2) results showing the interaction of CNX and ERp57 were confirmed, and the regions that promote this interaction defined.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A yeast two-hybrid assay for detection of interactions between at least two endoplasmic reticulum membrane and/or lumenal proteins of interest capable of or suspected of interacting, which comprises:
a) recombinational cloning of necessary DNA elements in a specially constructed plasmid in a reporter yeast strain wherein said plasmid is capable of expressing fusion proteins each comprising a fusion of a protein of interest and transmembrane domain and C-terminal kinase and RnaseL domains of yeast Ire1p kinase (unfolded protein response signaling kinase), wherein said reporter yeast strain having a reporter gene integrated and a Δirel genotype, wherein said reporter gene is controlled by an unfolded protein response (UPR) element, and wherein said reporter yeast strain non-recombined is incapable of an unfolded protein response (UPR); and
b) monitoring expression of said reporter gene by activation of an unfolded protein response (UPR), wherein said expression is indicative of protein interaction.

2. The assay of claim 1, wherein the reporter gene is LacZ gene from E. coli.

3. The assay of claim 2, wherein the reporter gene is controlled by a promoter having an unfolded protein response element upstream, and wherein said reporter is selected from the group consisting of HIS3, URA3, another yeast gene having a visible growth phenotype and a coloured substrate indicating transcription and translation of the reporter gene in response to oligomerization of Ire1p.

4. The assay of claim 3, wherein the promoter is a chimeric promoter comprising a minimal yeast unfolded protein response element (UPRE) and a truncated CYC1 promoter.

5. The assay of claim 1, wherein the protein interactions are occurring within an intracellular organelle.

6. The assay of claim 5, wherein the organelle is endoplasmic reticulum (ER).

7. The assay of claim 6, wherein the protein interactions are occurring within the endoplasmic reticulum (ER) lumen.

8. The assay of claim 1, wherein the protein is a membrane protein or a soluble protein.

9. The assay of claim 1, wherein the protein is a glycoprotein.

10. The assay of claim 1, wherein the yeast is Saccharomyces cerevisiae.

11. A method for mapping protein interactions, which comprises using the yeast two-hybrid assay according to claim 1.

12. The method of claim 12, wherein regions of protein-protein interaction of calnexin and calreticulin with Erp57 is mapped.

13. A high throughput assay for detection of protein interaction networks, which comprises the assay of claim 1 combined with high throughput technology.

14. A method for the analysis of protein interaction networks of the ER, which comprises using the assay of claim 13.
